(19) United States
(12) Patent Application Publication
(10) Pub. No.: US 2026/0193719 A1
(43) Pub. Date: Jul. 9, 2026

(54) QUANTIFYING MOLECULES BY A YEAST THREE HYBRID ASSAY

(71) Applicant: ANCHORLINE BIOLABS, INC., Dover, DE (US)

(72) Inventor: Dianna MAAR, Dover, DE (US)

(21) Appl. No.: 19/555,624

(22) Filed: Mar. 3, 2026

Related U.S. Application Data
(63) Continuation of application No. PCT/US2024/045588, filed on Sep. 6, 2024.
(60) Provisional application No. 63/580,890, filed on Sep. 6, 2023.

(51) Int. Cl. C12Q 1/6886; C12Q 1/6804; C12Q 1/6806; C12Q 1/6823; C12Q 1/6897

(52) U.S. Cl. CPC C12Q 1/6886; C12Q 1/6804; C12Q 1/6806; C12Q 1/6823; C12Q 1/6897

(57) ABSTRACT

The present disclosure describes a new method for quantifying a wide range of biological molecules. This disclosure sets forth systems and methods wherein a transcription activator protein is separated into two proteins, a DNA binding domain and an activation domain protein. These two proteins can be fused to or non-covalently attached to a molecule (such as a protein) known to bind to the quantified biological molecule (such as another protein). Bridging these two domains activates the transcriptional apparatus for transcription of a ribozyme. The transcribed ribozyme interacts with a probe to produce a signal for detection.

### CROSS-REFERENCE TO RELATED APPLICATIONS

This application is a continuation of PCT Application No. PCT/US2024/045588, filed Sep. 6, 2024, which claims priority to, and the benefit of, U.S. Provisional Ser. No. 63/580,890 , filed Sep. 6, 2023. The contents of the above-referenced applications are hereby incorporated by reference in their entireties for all purposes.

### FIELD

The present disclosure describes new methods, systems, and kits, for quantifying a wide range of biological molecules.

### BACKGROUND

Direct quantification of a biological molecule is helpful for understanding biological systems in which that molecule interacts. It remains a challenge to accurately and precisely measure certain biological molecules due to differences in testing sites, instruments, and users.

One method for quantifying biological molecules is quantitative polymerase chain reaction assays (qPCR). Digital PCR is a method for quantifying DNA and RNA. Digital PCR uses various partitioning methods and microfluidics to assign a quantity to a nucleic acid target from a relative qPCR score assigned. However, there are limitations to qPCR.

One method for quantifying proteins is the antibody-based Proximity Ligation Assays (PLA). This assay requires upstream preparation that often limits its use in assays that use partitions. Further, PLA includes washes or buffer exchanges that introduce variations in data due to sample mis-handling such as over-washing (producing false negative) or under-washing (producing false positives). Additionally, proximity ligation assays can create background noise that limits the ability to quantify low concentration samples of the target molecule.

Therefore, a need exists to accurately and reproducibly quantify molecules in biological assays.

### SUMMARY

The present disclosure describes methods, systems, and kits for quantifying a wide range of biological molecules. This disclosure describes methods and kits in which a transcription activator protein is separated into two proteins, a DNA binding domain and an activation domain protein. These protein domains can be fused to or non-covalently attached to biological molecules (e.g., proteins) known to bind to a target molecule. By bridging these two domains together, a transcriptional apparatus is activated to transcribe a ribozyme. The ribozyme in turn interacts with a probe allowing detection of a signal, which in turn allows quantification of the amount of target molecule.

In some embodiments, there is provided a method comprising bridging a first molecule domain with a second molecule domain by a target molecule; wherein the bridging activates transcription of one or more ribozymes. In some embodiments, there is provided a method for interacting a probe with the one or more ribozymes to produce an RNA readout. In some embodiments, the method comprises detecting the RNA readout.

In some embodiments, the method comprises partitioning a reaction mixture within a microfluidics device into partitions. In certain embodiments, the reaction mixture comprises one or more targets. In certain embodiments, the reaction mixture comprises one or more first molecule domains, the first molecule domains capable of interacting with one or more targets. In certain embodiments, the reaction mixture comprises one or more second molecule domains, the second molecule domains capable of interacting with one or more targets and one or more first molecule domains by bridging the first molecule domain to the second molecule domain, where bridging of the first molecule domain to the second molecule domain by interacting with the target, transcribes one or more ribozymes. In some embodiments, the method comprises reacting the reaction mixture to produce reacted partitions within the microfluidic device with one or more probes, wherein the one or more probes interact with one or more ribozymes to produce an RNA readout. In some embodiments, the method comprises detecting the RNA readout. In some embodiments, the method comprises quantifying the one or more target molecules from the reacted partitions.

In some embodiments, a kit is provided comprising a DNA sequence. In certain embodiments, the DNA sequence comprises an enhancer region, the enhancer region capable of interacting with a DNA binding domain. In certain embodiments, the DNA sequence comprises a transcriptional region, capable of interacting with a transcriptional activation domain, wherein the transcriptional region encodes a ribozyme. In some embodiments, the kit comprises one or more first molecule domains, capable of interacting with a target molecule and the enhancer region of the DNA sequence. In some embodiments, the kit comprises one or more second molecule domains, capable of interacting with a target molecule, and the transcriptional region. In some embodiments, the kit comprises one or more probes capable of interacting with one or more ribozyme to produce an RNA readout.

In some embodiments, a method is provided comprising bridging a first molecule domain with a second molecule domain by a target molecule; wherein the bridging activates transcription of a unique RNA sequence, wherein the sequence has a first adaptor sequence at the 5′ end and a second adaptor sequence at the 3′ end. In certain embodiments, the method comprises isolating the RNA transcript. In certain embodiments, the method comprises quantifying the RNA transcript.

In some embodiments, a method is provided comprising partitioning a reaction mixture within a microfluidic device into partitions. In certain embodiments, the reaction mixture comprises one or more target molecules. In certain embodiments, the reaction mixture comprises one or more first molecule domains, the first molecule domains capable of interacting with one or more target molecules. In certain embodiments, the reaction mixture comprises one or more second molecule domains, the second molecule domains capable of interacting with one or more target molecules and one or more first molecule domains by bridging the first molecule domain to the second molecule domain, where bridging of the first molecule domain to the second molecule domain by interacting with the target molecules, transcribes a unique RNA sequence, wherein the sequence has a first adaptor sequence at the 5′ end and a second adaptor sequence at the 3′ end. In certain embodiments, the method comprises isolating the RNA transcript. In certain embodiments, the method comprises detecting the RNA transcripts. In certain embodiments, the method comprises quantifying the target molecule from the reacted partitions.

Molecules other than DNA and RNA are quantified using relative measurement technologies. This relative measure is prone to gross error stemming from quantification, lab-to-lab variation, and day-to-day variation. The lack of accurate and reproducible quantification methods limits many molecules from being studied and developed as a therapy. Prior to this disclosure, converting other biological assays from a relative to a direct measurement for molecules other than DNA and/or RNA have had many challenges.

This disclosure describes methods and kits allowing for a wide-range of biological molecules to be quantified in the same devices used to perform digital PCR.

The methods and kits described herein can be used in a partitioning system for direct quantification of proteins and any molecule that can be bound by a protein or proteins including antibodies/aptamers.

The methods and kits described herein can be used to measure fully translated proteins, partially translated proteins, molecules that block molecule-molecule interactions, as well as other interactions between molecules.

The methods and kits described herein can be used in a sample partitioning system so that direct quantification of the molecule is made.

The methods and kits described herein have an advantage over conventional methods such as yeast-2-hybrid-like systems because the methods bypass the need for translational machinery. The methods described herein, enable a robust in vitro system for detecting and quantifying proteins and nucleic acids.

### BRIEF DESCRIPTIONS OF THE DRAWINGS

FIG. 1 depicts an embodiment of a method disclosed herein. An in vitro system for detecting and quantifying proteins is disclosed. A reaction mixture is made to contain a DNA binding domain protein (101) that is fused to a protein known to bind to a target molecule (102). The target molecule to be quantified (103), bridges the interaction with the transcription activation domain binding protein (104) that can then recruit the transcriptase (105) to the transcription start site (106) for transcription to occur. A ribozyme (107) reacting with a probe (108) creates an amplified signal that can be detected.

FIG. 2 depicts an embodiment of a method disclosed herein. An in vitro digital assay for quantifying a nucleic acid that is disclosed. A reaction mixture is made to contain a DNA binding domain protein (201) that is fused to a protein known to bind a target molecule (202). The target molecule to be quantified, shown as a ribonucleic acid, DNA, or RNA, of a specific sequence (203), bridges the interaction with the transcription activation domain binding protein (204) that is needed to recruit a transcriptase (205) to the transcription start site (206) for transcription to begin. A ribozyme (207) reacting with a probe (208) creates an amplified signal that can be detected.

FIG. 3 depicts an embodiment of a method disclosed herein. An in vitro digital assay for quantifying a protein is disclosed. The DNA binding domain protein (301) is fused to a protein known to bind to the Fc region of an antibody (302). This allows for a generic system that can be made to quantify different target molecules by adding an antibody that binds to the target molecule to be quantified (303). The antibody (302) that binds to the target molecule to be quantified (303) may be the same antibody, or may be different antibodies. Upon binding the target molecule, the system bridges the interaction with the transcription activation domain binding protein (304) that recruits the transcriptase (305) to the transcription start site (306) for transcription to begin. A ribozyme (307) reacting with a probe (308) creates an amplified signal that can be detected.

FIG. 4 depicts an embodiment of a method disclosed herein. The cleaving of a probe by a ribozyme is disclosed. One type of probe is an oligo nucleotide sequence with a fluorophore on one end and a quencher on the other (401). This probe has a sequence that allows it to bind to and become cleaved by the ribozyme (402). Upon cleaving the probe, the probe is split into at least two parts, wherein one part is attached to the fluorophore (403) and one part is attached to a quencher (404).

FIG. 5 depicts an embodiment of a method disclosed herein. Using a unique sequence that is flanked by P5 and P7 adaptor sequences, the RNA product can be isolated and detected allowing for a simplified system that excludes the need for translation machinery and chemistries to create a signal. The DNA binding domain protein (501) is fused to a protein (502) known to bind to a target molecule. The transcription activation domain (504) is fused to a protein (502) known to bind to a target molecule to be quantified (503). The protein (502) may be the same protein, or may be different proteins. The target molecule to be quantified (503), bridges the interaction with the transcription activation domain binding protein (504) that then recruits the transcriptase (505) to the transcription start site (506) for transcription to begin. A unique RNA sequence having P5 and P7 adaptors flanking it, is transcribed and can be isolated and detected to quantify a target molecule (507).

FIG. 6 depicts an embodiment of a method disclosed herein. Using a unique sequence that is flanked by P5 and P7 adaptor sequences, the RNA product can be isolated and sequenced allowing for a simplified system that excludes the need for translation machinery and chemistries to create a signal. This simplification enables a robust in vitro system for detecting and quantifying nucleic acids. The DNA binding domain protein (601) is fused to a protein known to bind to your target molecule such as an DNA or RNA (602).. The transcription activation domain binding protein (604) is fused to a protein (602) known to bind to a target molecule to be quantified (603). The protein (602) may be the same protein, or may be different proteins. The target molecule to be quantified (603), bridges the interaction with the transcription activation domain binding protein (604) that then recruits the transcriptase (605) to the transcription start site (606) for transcription to occur. A unique RNA sequence having P5 and P7 adaptors flanking it, is transcribed and can be isolated and detected to quantify a target molecule (507).

### DETAILED DESCRIPTION

The following description is presented to enable one of ordinary skill in the art to make and use the disclosed subject matter and to incorporate it in the context of applications. Various modifications, as well as a variety of uses in different applications, will be readily apparent to those skilled in the art, and the general principles defined herein may be applied to a wide range of embodiments. Thus, the present disclosure is not intended to be limited to the embodiments presented, but is to be accorded the widest scope consistent with the principles and novel features disclosed herein.

### Definitions

As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “oligonucleotide” refers to a polymer or oligomer of nucleotide monomers, containing any combination of nucleobases, modified nucleobases, sugars, modified sugars, phosphate bridges, or modified bridges.

Oligonucleotides can be of various lengths. In particular embodiments, oligonucleotides can range from about 2 to about 500 nucleotides in length. In various related embodiments, oligonucleotides, single-stranded, double-stranded, and triple-stranded, can range in length from about 12 to about 20 nucleotides, from about 10 to about 60 nucleotides, from about 10 to about 90 nucleotides, from about 15 to about 30 nucleotides, from about 20 to about 120 nucleotides in length. In some embodiments, the oligonucleotide is from about 4 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length.

As used herein, the term “probe” or “probes” refers to any molecules, synthetic or naturally occurring that is in need of detection. For example, a probe can include a nucleic acid molecule, an oligonucleotide, a protein, a peptide, or combinations thereof. For example, a protein probe may be connected with one or more nucleic acid molecules to form a probe that is a chimera. As disclosed herein, in some embodiments, a probe itself can produce a detectable signal. In some embodiments, a probe is connected, directly or indirectly with a signal moiety (e.g., a dye or fluorophore) that can produce a detectable signal.

As used herein, the term “sample” refers to a biological sample obtained or derived from a source of interest, as described herein. In some embodiments, a source of interest comprises an organism, such as an animal, plant, microorganism or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the term “sample” refers to a nucleic acid such as DNA, RNA, transcripts, or chromosomes. In some embodiments, the term “sample” refers to nucleic acid that has been extracted from the cell.

As disclosed herein, the term “binding” refers to the interaction of two molecules. In certain embodiments, “binding” may refer to the hybridization of two nucleotide sequences. In certain embodiments, “binding” may refer to a protein-nucleotide interaction. In certain embodiments, “binding” may refer to a protein-protein interaction.

As defined herein, the term “close proximity” refers to the distance between two objects wherein the first object is about 5, 10, 20, 30, 40, 50, 60, 70, 80, 90, 100, 150, 200, 250, 300, 350, 400, 450, 500, 550, 600, 650, 700, 750, 800, 850, 900, 950, 1000, 1500, 2000, or 3000 nanometers from the other.

As defined herein, the term “interacting” refers to physical interaction between two molecules. These interactions comprise the physical interaction between protein, RNA, DNA, and molecular targets. These physical interactions may use at least one of: hydrogen bonding, electrostatic interactions, covalent bonding, hydrophobic bonding, van der Waals forces, and salt bridges to form the interactions.

As defined herein, the term “fluorophore” refers to any material or molecule that can re-emit light upon light excitation.

As defined herein, the term “quencher” refers to any material or molecule that can absorb energy from a fluorophore and re-emit much of that energy as either heat (in the case of dark quenchers) or visible light (in the case of fluorescent quenchers).

### Overview

In some embodiments, there is provided a method comprising bridging a first molecule domain with a second molecule domain by a target molecule; wherein the bridging activates transcription of one or more ribozymes. In some embodiments, there is provided a method for interacting a probe with the one or more ribozymes to produce an RNA readout. In some embodiments, the method comprises detecting the RNA readout.

In certain embodiments, the method comprises adding the target molecule to a reaction mixture comprising a first molecule domain and a second molecule domain.

In certain embodiments, the method comprises quantifying the RNA readout to determine a quantity of the target molecule.

In some embodiments, the method comprises partitioning a reaction mixture within a microfluidics device into partitions. In certain embodiments, the reaction mixture comprises one or more targets. In certain embodiments, the reaction mixture comprises one or more first molecule domains, the first molecule domains capable of interacting with one or more targets. In certain embodiments, the reaction mixture comprises one or more second molecule domains, the second molecule domains capable of interacting with one or more targets and one or more first molecule domains by bridging the first molecule domain to the second molecule domain, where bridging of the first molecule domain to the second molecule domain by interacting with the target, transcribes one or more ribozymes. In some embodiments, the method comprises reacting the reaction mixture to produce reacted partitions within the microfluidic device with one or more probes, wherein the one or more probes interact with one or more ribozymes to produce an RNA readout. In some embodiments, the method comprises detecting the RNA readout. In some embodiments, the method comprises quantifying the one or more target molecules from the reacted partitions.

In certain embodiments, the method further comprises collecting the reacted partitions from the microfluidic device.

In certain embodiments, the method further comprises quantifying the ribozymes as a readout to determine the quantity of target molecules. In certain embodiments, the ribozymes are quantified by collecting the ribozymes. In certain embodiments, the collected ribozymes are quantified using techniques selected from PCR, Next Generation Sequencing, gel electrophoresis, and any technology used for RNA quantification known to a person of skill.

In some embodiments, a kit is provided comprising a DNA sequence. In certain embodiments, the DNA sequence comprises an enhancer region, the enhancer region capable of interacting with a DNA binding domain. In certain embodiments, the DNA sequence comprises a transcriptional region, capable of interacting with a transcriptional activation domain, wherein the transcriptional region encodes a ribozyme. In some embodiments, the kit comprises one or more first molecule domains, capable of interacting with a target molecule and the enhancer region of the DNA sequence. In some embodiments, the kit comprises one or more second molecule domains, capable of interacting with a target molecule, and the transcriptional region. In some embodiments, the kit comprises one or more probes capable of interacting with one or more ribozyme to produce an RNA readout.

In some embodiments, a method is provided comprising bridging a first molecule domain with a second molecule domain by a target molecule; wherein the bridging activates transcription of a unique RNA sequence, wherein the sequence has a first adaptor sequence at the 5′ end and a second adaptor sequence at the 3′ end. In certain embodiments, the method comprises isolating the RNA transcript. In certain embodiments, the method comprises quantifying the RNA transcript.

In some embodiments, a method is provided comprising partitioning a reaction mixture within a microfluidic device into partitions. In certain embodiments, the reaction mixture comprises one or more target molecules. In certain embodiments, the reaction mixture comprises one or more first molecule domains, the first molecule domains capable of interacting with one or more target molecules. In certain embodiments, the reaction mixture comprises one or more second molecule domains, the second molecule domains capable of interacting with one or more target molecules and one or more first molecule domains by bridging the first molecule domain to the second molecule domain, where bridging of the first molecule domain to the second molecule domain by interacting with the target molecules, transcribes a unique RNA sequence, wherein the sequence has a first adaptor sequence at the 5′ end and a second adaptor sequence at the 3′ end. In certain embodiments, the method comprises isolating the RNA transcript. In certain embodiments, the method comprises detecting the RNA transcripts. In certain embodiments, the method comprises quantifying the target molecule from the reacted partitions.

### Molecular Targets

In some embodiments, the method comprises one or more molecular targets.

In certain embodiments, the molecular targets comprise extracts from samples. In certain embodiments, the samples are obtained from patients.

In some embodiments, the molecular targets are selected from proteins, modified proteins, transcripts, RNA, DNA loci, exogenous proteins, exogenous nucleic acids, hormones, carbohydrates, small molecules, biologically active molecules, and combinations thereof. In some embodiments, the molecular targets are proteins. In some embodiments, the molecular targets are modified proteins. In some embodiments, the molecular targets are transcripts. In some embodiments, the molecular targets are RNA molecules. In some embodiments, the molecular targets are DNA loci. In some embodiments, the molecular targets are exogenous proteins. In some embodiments, the molecular targets are exogenous nucleic acids. In some embodiments, the molecular targets are hormones. In some embodiments, the molecular targets are carbohydrates. In some embodiments, the molecular targets are small molecules. In some embodiments, the molecular targets are biologically active molecules.

In some embodiments, molecular targets comprise Viral capsid proteins (AAV capsids, Lentivirus capsids), THC, Psilocybin, prions, Glycans, metabolites, lipids, protein-protein interactions, or protein-DNA interactions.

### First Molecule Domain and Second Molecule Domain

In certain embodiments, the reaction mixture comprises one or more first molecule domains, the first molecule domains capable of interacting with one or more targets.

In some embodiments, the method comprises a first molecule domain that is linked to a DNA binding domain capable of interacting with an enhancer region. In certain embodiments, the enhancer region is capable of activating transcription of a gene encoding a ribozyme. In certain embodiments, the method comprises a first molecule domain that is linked to the DNA binding domain by a polypeptide.

In some embodiments, the method comprises a second molecule domain that is linked to a transcriptional activation domain capable of interacting with a transcriptional region on DNA sequence. In certain embodiments, the method comprises a second molecule domain that is linked to a transcriptional activation domain by a polypeptide.

In some embodiments, the first binding domain, the second binding domain, or both, bind to an antibody, aptamer, or combination thereof. In certain embodiments, the antibody, aptamer, or combination thereof bound to the first binding domain, the second binding domain, or both, interact with the target. In certain embodiments, the antibody, aptamer, or combination thereof bound to the first binding domain is the same as the antibody, aptamer, or combination thereof bound to the second binding domain. In certain embodiments, the antibody, aptamer, or combination thereof bound to the first binding domain is different than the antibody, aptamer, or combination thereof bound to the second binding domain. In certain embodiments, the antibodies, aptamers, or combinations thereof are directly fused to the first binding domain, the second binding domain, or both. In certain embodiments, the antibodies, aptamers, or combination thereof are linked to the first binding domain, second binding domain, or both, by an avidin and streptavidin interaction.

In some embodiments, the method comprises a first binding domain that is an antibody, aptamer, or combination thereof. In some embodiments, the method comprises a second binding domain that is an antibody, aptamer, or combination thereof.

In some embodiments, the first binding domain, the second binding domain, or both, bind to an antibody, aptamer, peptide ligands, nucleic acid mimics, molecular imprinted polymers, affibodies, synthetic antibody mimics, lectins, adnectins, anticalins, lipid binding proteins, molecules that can specifically bind targets of interest, or combinations thereof. In certain embodiments, the antibody, aptamer, peptide ligands, nucleic acid mimics, molecular imprinted polymers, affibodies, synthetic antibody mimics, lectins, adnectins, anticalins, lipid binding proteins, molecules that can specifically bind targets of interest, or combinations thereof bound to the first binding domain, the second binding domain, or both, interact with the target. In certain embodiments, the antibody, aptamer, peptide ligands, nucleic acid mimics, molecular imprinted polymers, affibodies, synthetic antibody mimics, lectins, adnectins, anticalins, lipid binding proteins, molecules that can specifically bind targets of interest, or combinations thereof bound to the second binding domain. In certain embodiments, the antibody, aptamer, peptide ligands, nucleic acid mimics, molecular imprinted polymers, affibodies, synthetic antibody mimics, lectins, adnectins, anticalins, lipid binding proteins, molecules that can specifically bind targets of interest, or combinations thereof bound to the second binding domain.

In certain embodiments, the antibody, aptamer, peptide ligands, nucleic acid mimics, molecular imprinted polymers, affibodies, synthetic antibody mimics, lectins, adnectins, anticalins, lipid binding proteins, molecules that can specifically bind targets of interest, or combinations thereof are directly fused to the first binding domain, the second binding domain, or both.

In certain embodiments, the antibody, aptamer, peptide ligands, nucleic acid mimics, molecular imprinted polymers, affibodies, synthetic antibody mimics, lectins, adnectins, anticalins, lipid binding proteins, molecules that can specifically bind targets of interest, or combinations thereof are linked to the first binding domain, second binding domain, or both, by an avidin and streptavidin interaction.

In some embodiments, the method comprises a first binding domain that is an antibody, aptamer, peptide ligands, nucleic acid mimics, molecular imprinted polymers, affibodies, synthetic antibody mimics, lectins, adnectins, anticalins, lipid binding proteins, molecules that can specifically bind targets of interest, or combinations thereof. In some embodiments, the method comprises a second binding domain that is an antibody, aptamer, peptide ligands, nucleic acid mimics, molecular imprinted polymers, affibodies, synthetic antibody mimics, lectins, adnectins, anticalins, lipid binding proteins, molecules that can specifically bind targets of interest, or combinations thereof.

In some embodiments, the first molecule domain, second molecule domain, or both interacts with the molecular target with a K_{D} of less than 100 nM. In some embodiments, the first molecule domain, second molecule domain, or both interacts with the molecular target with a K_{D} of less than 1000 nM. In some embodiments, the first molecule domain, second molecule domain, or both interacts with a molecular target with a K_{D} of less than 900 nM. In some embodiments, the first molecule domain, second molecule domain, or both interacts with the molecular target with a K_{D} of less than 800 nM. In some embodiments, the first molecule domain, second molecule domain, or both interacts with the molecular target with a K_{D} of less than 700 nM. In some embodiments, the first molecule domain, second molecule domain, or both interacts with the molecular target with a K_{D} of less than 600 nM. In some embodiments, the first molecule domain, second molecule domain, or both interacts with the molecular target with a K_{D} of less than 500 nM. In some embodiments, the first molecule domain, second molecule domain, or both interacts with the molecular target with a K_{D} of less than 400 nM. In some embodiments, the first molecule domain, second molecule domain, or both interacts with the molecular target with a K_{D} of less than 300 nM. In some embodiments, the first molecule domain, second molecule domain, or both interacts with the molecular target with a K_{D} of less than 200 nM.

In some embodiments, the K_{D} of any of the previous embodiments is measured by surface plasmon resonance techniques with the target.

In some embodiments, the first binding domain is linked to a DNA binding domain capable of interacting with an enhancer region on DNA sequence capable of transcribing a ribozyme.

In some embodiments, the second binding domain is linked to a transcriptional activation domain capable of interacting with a transcriptional region on DNA sequence capable of transcribing a ribozyme.

### RNA Transcript and Ribozyme

In some embodiments, the method comprises transcribing a ribozyme.

In some embodiments, the ribozyme is selected from a GIR1 branching ribozyme, glmS ribozyme, Group I self-splicing intron, Group II self-splicing intron—Spliceosome is likely derived from Group II self-splicing ribozymes, Hairpin ribozyme, Hammerhead ribozyme, HDV ribozyme, rRNA, RNase P, Twister ribozyme, Twister sister ribozyme, VS ribozyme, Pistol ribozyme, Hatchet ribozyme, Viroids, Deoxyribozyme, or an artificial ribozyme.

In some embodiments, the method comprises one or more ribozymes that are transcribed, wherein each probe capable of being cleaved by ribozyme is cleaved by a specific ribozyme for that probe.

In some embodiments, the method comprises transcribing a unique RNA sequence.

In some embodiments, the unique RNA sequence is less than 5000 nucleotides long. In some embodiments, the unique RNA sequence is less than 4000 nucleotides long. In some embodiments, the unique RNA sequence is less than 3000 nucleotides long. In some embodiments, the unique RNA sequence is less than 2000 nucleotides long. In some embodiments, the unique RNA sequence is less than 1000 nucleotides long. In some embodiments, the unique RNA sequence is less than 900 nucleotides long. In some embodiments, the unique RNA sequence is less than 800 nucleotides long. In some embodiments, the unique RNA sequence is less than 700 nucleotides long. In some embodiments, the unique RNA sequence is less than 600 nucleotides long. In some embodiments, the unique RNA sequence is less than 500 nucleotides long. In some embodiments, the unique RNA sequence is less than 400 nucleotides long. In some embodiments, the unique RNA sequence is less than 300 nucleotides long. In some embodiments, the unique RNA sequence is less than 200 nucleotides long. In some embodiments, the unique RNA sequence is less than 100 nucleotides long.

In certain embodiments, the unique RNA sequence of any of the previous embodiments is at least 12 nucleotides long. In certain embodiments, the unique RNA sequence of any of the previous embodiments is at least 15 nucleotides long. In certain embodiments, the unique RNA sequence of any of the previous embodiments is at least 20 nucleotides long. In certain embodiments, the unique RNA sequence of any of the previous embodiments is at least 25 nucleotides long. In certain embodiments, the unique RNA sequence of any of the previous embodiments is at least 30 nucleotides long. In certain embodiments, the unique RNA sequence of any of the previous embodiments is at least 35 nucleotides long. In certain embodiments, the unique RNA sequence of any of the previous embodiments is at least 40 nucleotides long. In certain embodiments, the unique RNA sequence of any of the previous embodiments is at least 50 nucleotides long. In certain embodiments, the unique RNA sequence of any of the previous embodiments is at least 60 nucleotides long. In certain embodiments, the unique RNA sequence of any of the previous embodiments is at least 70 nucleotides long. In certain embodiments, the unique RNA sequence of any of the previous embodiments is at least 80 nucleotides long. In certain embodiments, the unique RNA sequence of any of the previous embodiments is at least 90 nucleotides long. In certain embodiments, the unique RNA sequence of any of the previous embodiments is at least 100 nucleotides long.

In certain embodiments, a first adaptor sequence is attached to the 5′ end of the unique RNA sequence. In certain embodiments, a second adaptor sequence is attached to the 3′ end of the unique RNA sequence. In certain embodiments, the first adaptor sequence and the second adaptor sequence are the same. In certain embodiments, the first adaptor sequence and the second adaptor sequence are different. In certain embodiments, the first or second adaptor sequence is the P5 sequence. In certain embodiments, the first or second adaptor sequence is the P7 sequence. In certain embodiments, the first or second adaptor sequence is selected from any of the previous embodiments.

In some embodiments, the adaptor sequence is less than 5000 nucleotides long. In some embodiments, the adaptor sequence is less than 4000 nucleotides long. In some embodiments, the adaptor sequence is less than 3000 nucleotides long. In some embodiments, the adaptor sequence is less than 2000 nucleotides long. In some embodiments, the adaptor sequence is less than 1000 nucleotides long. In some embodiments, the adaptor sequence is less than 900 nucleotides long. In some embodiments, the adaptor sequence is less than 800 nucleotides long. In some embodiments, the adaptor sequence is less than 700 nucleotides long. In some embodiments, the adaptor sequence is less than 600 nucleotides long. In some embodiments, the adaptor sequence is less than 500 nucleotides long. In some embodiments, the adaptor sequence is less than 400 nucleotides long. In some embodiments, the adaptor sequence is less than 300 nucleotides long. In some embodiments, the adaptor sequence is less than 200 nucleotides long. In some embodiments, the adaptor sequence is less than 100 nucleotides long.

In certain embodiments, the adaptor sequence of any of the previous embodiments is at least 15 nucleotides long. In certain embodiments, the adaptor sequence of any of the previous embodiments is at least 20 nucleotides long. In certain embodiments, the adaptor sequence of any of the previous embodiments is at least 25 nucleotides long. In certain embodiments, the adaptor sequence of any of the previous embodiments is at least 30 nucleotides long. In certain embodiments, the adaptor sequence of any of the previous embodiments is at least 35 nucleotides long. In certain embodiments, the adaptor sequence of any of the previous embodiments is at least 40 nucleotides long. In certain embodiments, the adaptor sequence of any of the previous embodiments is at least 50 nucleotides long. In certain embodiments, the adaptor sequence of any of the previous embodiments is at least 60 nucleotides long. In certain embodiments, the adaptor sequence of any of the previous embodiments is at least 70 nucleotides long. In certain embodiments, the adaptor sequence of any of the previous embodiments is at least 80 nucleotides long. In certain embodiments, the adaptor sequence of any of the previous embodiments is at least 90 nucleotides long. In certain embodiments, the adaptor sequence of any of the previous embodiments is at least 100 nucleotides long.

In some embodiments, the method of any of the previous embodiments, comprises transcribing a unique RNA that hybridizes with an oligonucleotide. In certain embodiments, the unique RNA hybridized to an oligonucleotide is cleaved by an enzyme. In certain embodiments, the enzyme is RNaseH.

In some embodiments, the method of any of the previous embodiments, comprises transcribing a ribozyme that hybridizes with an oligonucleotide. In certain embodiments, the ribozyme hybridized to an oligonucleotide is cleaved by an enzyme. In certain embodiments, the enzyme is RNaseH.

### Probes and Readout

In some embodiments, the method comprises a transcribed ribozyme that interacts with a probe to produce an RNA readout. In some embodiments, the interaction of the probe with one or more ribozymes comprises a ligation, cleavage, binding, chemical modification, or any combination thereof of the probe to produce an RNA readout. In certain embodiments, the ribozyme cleaves the probe to produce an RNA readout.

In some embodiments, the probes are selected from an oligonucleotide, peptide, or combination thereof. In certain embodiments, the probes are oligonucleotides.

In some embodiments, the probes comprise oligonucleotides are at least 5, 10, 15, 20, 25, 30, 35, 40, 45, 50, 55, 60, 70, 80, 90, or 100 nucleotides long.

In some embodiments, the RNA readout is a fluorescent readout.

In some embodiments, the fluorophore is any fluorophore deemed suitable by those of skill in the arts. In some embodiments, the fluorophores include but are not limited to fluorescein, rhodamine, Alexa Fluors, DyLight fluors, ATTO Dyes, or any analogs or derivatives thereof. In certain embodiments, the detectable moieties include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red).

In some embodiments, the oligonucleotide has a fluorophore at the 5′ end, 3′ end, between the 5′ and 3′ ends, or any combination thereof. In some embodiments, the quencher has a fluorophore at the 5′ end, 3′ end, between the 5′ and 3′ ends, or any combination thereof.

In some embodiments, the fluorophore is a nanoparticle. In some embodiments, the fluorophore is a quantum dot. In some embodiments, the fluorophore is a gold particle. In some embodiments, the quencher is a nanoparticle. In some embodiments, the quencher is a quantum dot. In some embodiments, the quencher is a gold particle.

In some embodiments, the quencher is a nanoparticle. In some embodiments, the quencher is a quantum dot. In some embodiments, the quencher is a gold particle.

### Partitioning the Reaction Mixture

In some embodiments, the method comprises partitioning the reaction mixture. As understood by a person having ordinary skill in the art, different technologies can be used for the partitioning of the reaction mixture.

In some embodiments, the method partitions a reaction mixture. In certain embodiments, the reaction mixture is a bulk reaction that is not partitioned.

### Detection

In some embodiments, the method comprises imaging the detectably labelled probes. In some embodiments, the method comprises imaging the probes. As understood by a person having ordinary skill in the art, different technologies can be used for the imaging steps.

In some embodiments, the method of detection uses quantitative PCR. In some embodiments, the method of detection uses a charge coupled device (CCD) to measure the fluorescent readout. In some embodiments, the method of detection uses a fluorimeter. In some embodiments, the method of detection uses light microscopy. In some embodiments, the method of detection uses fluorescent microscopy.

In some embodiments, the method comprises quantifying the probe. In certain embodiments, the probe is quantified using quantitative PCR (qPCR). In certain embodiments, the method of detection comprises quantifying the amount of ribozymes produced. In certain embodiments, the method comprises detecting a unique RNA sequence. In certain embodiments, the method of detection comprises detecting the RNA produced by any of the previous embodiments, by using digital PCR, qPCR, gel electrophoresis, next generation sequencing, or any method used for quantifying RNA.

In some embodiments, the method of detection detects a probe acted upon by a ribozyme to create a signal. In certain embodiments, the method of detection uses digital PCR (dPCR) instruments. In certain embodiments the method of detection uses quantitative PCR (qPCR) instruments. In certain embodiments, the method of detection uses photomultiplier tube (PMT) equipped devices. In certain embodiments, fluorescence generated by the method is detected via laser excitation. In certain embodiments, the laser excitation is detected with appropriate wavelength emission filters and either a PMT or avalanche photodiode.

In some embodiments, the method of detection uses sequencing technologies. In certain embodiments, the sequencing technology is sanger sequencing. In certain embodiments, the sequencing technology is a Next Generation Sequencing (NGS) platforms.

In some embodiments, the method of detection uses gel electrophoresis. In certain embodiments, the gel electrophoresis is an agarose or polyacrylamide gel.

In some embodiments, the method of detection uses a colorimetric change of substrate that is proportional to an amplified signal produced by the method. In certain embodiments, the method of detection uses a pH change as a result of DNA synthesis during an amplification process of the assay to release protons.

In some embodiments, the method of detection uses a microfluidic device in combination with any of the previous embodiments.

In some embodiments, the method comprises detecting single target molecules.

In some embodiments, the method comprises detecting single target molecules by applying Poisson statistics.

### Diseases and Conditions

In some embodiments, the method comprises a method of diagnosis of a disease or condition in a subject in need thereof comprising any of the previous embodiments to detect the target molecules indicating the disease or condition in the subject.

In some embodiments, the method comprises treating a disease or condition in a subject in need thereof comprising using any of the previous embodiments to detect the target molecules indicating the disease or condition in the subject.

In some embodiments, the method comprises maintaining a health condition in a subject in need thereof comprising use of any of the previous embodiments to detect the target molecules indicating the disease or condition in a subject.

In certain embodiments, the disease or condition comprises diseases caused by prions, cancer, heart disease, genetic diseases, or combinations thereof. In certain embodiments, the disease or condition is cancer. In certain embodiments, the disease or condition is heart disease.

### Kits

In some embodiments, any of the previous embodiments are used in kits.

In some embodiments, the kits comprise one or more target molecules as a positive control. In some embodiments, the kits comprise one or more target molecules as a negative control.

### EXAMPLES

Those of skill in the art will understand that the drawings, described below, are for illustrative purposes only. The drawings are not intended to limit the scope of the present teachings in any way.

### Example 1—Prophetic

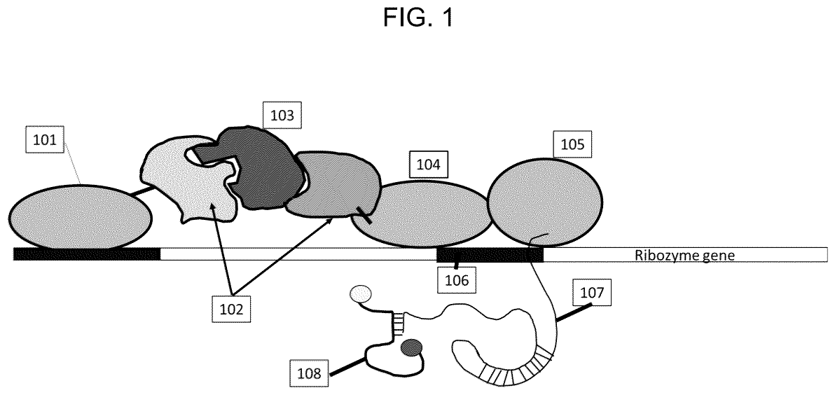

This example, as illustrated in FIG. 1, depicts an exemplary design of the method.

A reaction mixture would be prepared that allows for the transcription of a ribozyme gene encoded on a plasmid. The reaction mixture would comprise a transcriptase, MgCl₂, ribonucleotides, and Tris-HCL buffer. The reaction mixture would be incubated at 37° C. The reaction further would comprise a probe that functions as a readout probe. The readout probe would be an oligonucleotide with a fluorescence moiety on the 5′ end and quencher moiety on the 3′ end.

A recombinant binding domain (BD)-X fused protein would be added to the reaction mixture. The BD-X protein would comprise a binding domain (BD) (101) that interacts with an enhancer sequence on a plasmid. The X domain of the BD-X fusion protein would comprise a protein (102) capable of interacting with a target molecule.

An activation domain (AD)-Y fused protein would be added to the reaction mixture. The reaction mixture would comprise a transcriptional activation domain (AD) (104) that interacts with a transcriptional start site on the plasmid (106) and would be capable of recruiting a transcriptase (105). The Y domain of the AD-Y fusion protein would comprise a protein (102) capable of interacting with a target molecule.

Upon adding the target molecule (103), the X domain and the Y domain would be bridged. This bridging would activate a transcriptase that begins transcribing a ribozyme (107).

The probe (108) in the reaction mixture would interact with the ribozyme. In this case, the ribozyme would cleave the probe allowing the quencher to be separated from the probe and detection of the fluorophore attached to the remnants of the probe by a digital PCR device.

### Example 2—Prophetic Example

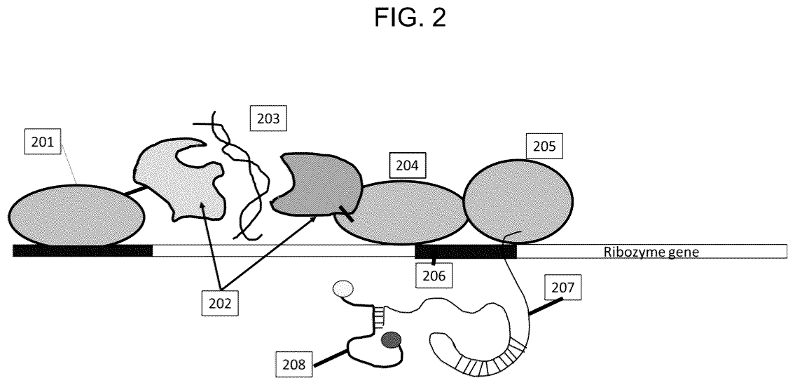

This example, as illustrated in FIG. 2, depicts an exemplary design of the method.

A reaction mixture would be prepared that allows for the transcription of a ribozyme gene encoded on a plasmid. The reaction mixture would comprise a transcriptase, MgCl₂, ribonucleotides, and Tris-HCL buffer. The reaction mixture would be incubated at 37° C. The reaction further would comprise a probe that functions as a readout probe. The readout probe would be an oligonucleotide with a fluorescence moiety on the 5′ end and quencher moiety on the 3′ end.

A recombinant binding domain (BD)-X fused protein would be added to the reaction mixture. The BD-X protein would comprise a binding domain (BD) (201) that interacts with an enhancer sequence on a plasmid. The X domain of the BD-X fusion protein would comprise a protein (202) capable of interacting with a target molecule.

An activation domain (AD)-Y fused protein would be added to the reaction mixture. The reaction mixture would comprise a transcriptional activation domain (AD) (204) that interacts with a transcriptional start site (206) on the plasmid and would be capable of recruiting a transcriptase (205). The Y domain of the AD-Y fusion protein would comprise a protein (202) capable of interacting with a target molecule.

Upon adding the target molecule (203), the X domain and the Y domain would be bridged. The target molecule in this example would comprise DNA. This bridging would activate a transcriptase that begins transcribing a ribozyme (207).

The probe (208) in the reaction mixture would interact with the ribozyme. In this case, the ribozyme would cleave the probe allowing the quencher to be separated from the probe and detection of the fluorophore attached to the remnants of the probe by a digital PCR device.

### Example 3—Prophetic Example

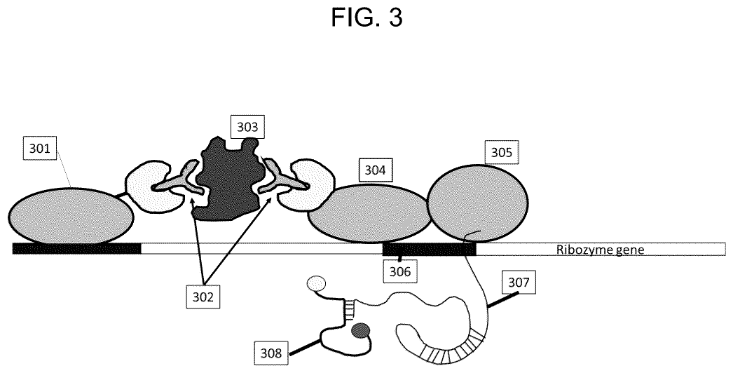

This example, as illustrated in FIG. 3, depicts an exemplary design of the method. In this exemplary design a protein serves as the target molecule and is recognized through the binding of antibodies.

A reaction mixture would be prepared that allows for the transcription of a ribozyme gene encoded on a plasmid. The reaction mixture would comprise a transcriptase, MgCl₂, ribonucleotides, and Tris-HCL buffer. The reaction mixture would be incubated at 37° C. The reaction further would comprise a probe that functions as a readout probe. The readout probe would be an oligonucleotide with a fluorescence moiety on the 5′ end and quencher moiety on the 3′ end.

A recombinant binding domain (BD)-X fused protein would be added to the reaction mixture. The BD-X protein would comprise a binding domain (BD) (301) that interacts with an enhancer sequence on a plasmid. The X domain of the BD-X fusion protein would comprise a protein capable of interacting with a target molecule via an antibody. The X (302) domain of the BD-X fusion protein would be bound to an antibody's constant domain. This would allow the antibody's variable domain to interact with the protein molecular target.

An activation domain (AD)-Y fused protein would be added to the reaction mixture. The reaction mixture would comprise a transcriptional activation domain (AD) (304) that interacts with a transcriptional start site (306) on the plasmid and would be capable of recruiting a transcriptase (305). The Y domain of the AD-Y fusion protein would comprise a protein capable of interacting with a target molecule. The Y domain (302) of the AD-Y fusion protein would be bound to an antibody's constant domain. This would allow the antibody's variable domain to interact with the protein molecular target.

Upon adding a protein target (303), the antibodies that were bound to the X domain and the Y domain would be bridged. This bridging would activate a transcriptase that begins transcribing a ribozyme (307).

The probe (308) in the reaction mixture would interact with the ribozyme. In this case, the ribozyme would cleave the probe allowing the quencher to be separated from the probe and detection of the fluorophore attached to the remnants of the probe by a digital PCR device.

### Example 4—Prophetic Example

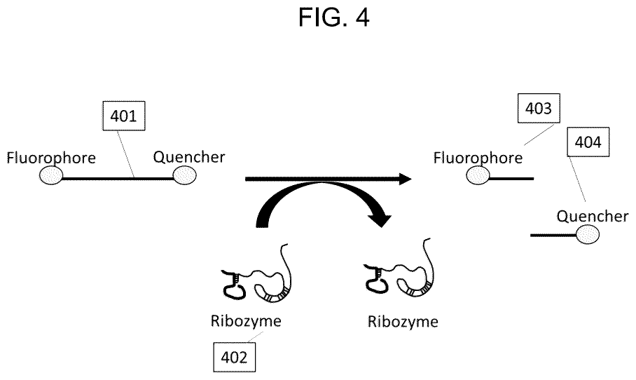

FIG. 4 depicts an exemplary design of a ribozyme cleaving an RNA probe.

In this exemplary design a DNA probe (401) that would have a fluorophore on the 5′ end and quencher on the 3′ end would be cleaved by a ribozyme. Before cleavage of the DNA probe, there would be no signal that could be detected by measuring the fluorescence. This is because, the quencher would negate the fluorescent signal. Once the probe interacts with the ribozyme, the probe would be cleaved (402). The effect of the quencher would be negated and a fluorescent signal on the remaining probe would be detected.

### Example 5—Prophetic Example

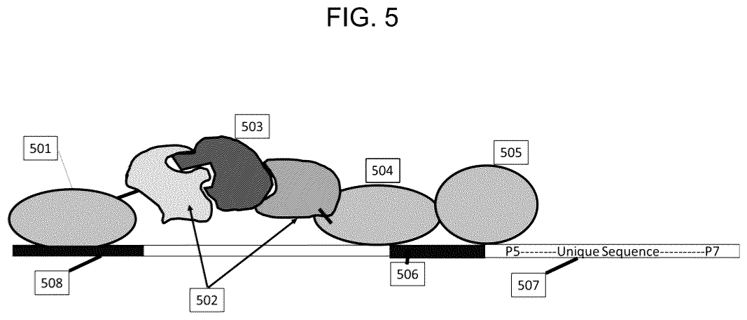

This example, as illustrated in FIG. 5, depicts an exemplary design of the method.

A reaction mixture would be prepared that allows for the transcription of a ribozyme gene encoded on a plasmid. The reaction mixture would comprise a transcriptase, MgCl2, ribonucleotides, and Tris-HCL buffer. The reaction mixture would be incubated at 37° C. The reaction further would comprise a probe that functions as a readout probe. The readout probe would be an oligonucleotide with a fluorescence moiety on the 5′ end and quencher moiety on the 3′ end.

A recombinant binding domain (BD)-X fused protein would be added to the reaction mixture. The BD-X protein would comprise a binding domain (BD) (601) that interacts with an enhancer sequence on a plasmid. The X domain of the BD-X fusion protein would comprise a protein capable of interacting with a target molecule (602).

The DNA binding domain (501) and the X-domain (502) would be unique for each target molecule to be detected.

An activation domain (AD)-Y fused protein would be added to the reaction mixture. The reaction mixture would comprise a transcriptional activation domain (AD) (604) that interacts with a transcriptional start site (506) on the plasmid and would be capable of recruiting a transcriptase (505). The Y domain of the AD-Y fusion protein would comprise a protein capable of interacting with a target molecule (502).

Upon adding the target molecule (503), the X domain and the Y domain would be bridged. This bridging would activate a transcriptase that begins transcribing a unique RNA sequence (507) that would identify a particular target molecule. The unique sequence would correspond to a target molecule. The unique RNA sequence would have P5 and P7 adaptor sequences flanking the sequence. The transcripts would be collected and analyzed to detect their presence, allowing quantification of the target molecule.

The P5 and P7 adaptor sequences would bind to the flow cell of the Illumina platform, allowing for the bridge amplification needed for sequencing. This would allow detection and quantification of the molecular target.

### Example 6—Prophetic Example

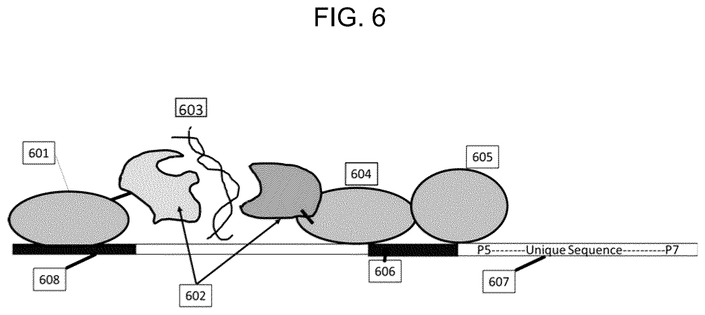

This example, as illustrated in FIG. 6, depicts an exemplary design of the method.

A reaction mixture would be prepared that allows for the transcription of a ribozyme gene encoded on a plasmid. The reaction mixture would comprise a transcriptase, MgCl₂, ribonucleotides, and Tris-HCL buffer. The reaction mixture would be incubated at 37° C. The reaction further would comprise a probe that functions as a readout probe. The readout probe would be an oligonucleotide with a fluorescence moiety on the 5′ end and quencher moiety on the 3′ end.

A recombinant binding domain (BD)-X fused protein would be added to the reaction mixture. The BD-X protein would comprise a binding domain (BD) (601) that interacts with an enhancer sequence on a plasmid. The X domain of the BD-X fusion protein would comprise a protein capable of interacting with a target molecule (602).

The DNA binding domain (601) and the X-domain (602) would be unique for each target molecule to be detected.

An activation domain (AD)-Y fused protein would be added to the reaction mixture. The reaction mixture would comprise a transcriptional activation domain (AD) (604) that interacts with a transcriptional start site (606) on the plasmid and would be capable of recruiting a transcriptase (605). The Y domain of the AD-Y fusion protein would comprise a protein capable of interacting with a target molecule (602).

Upon adding the target molecule (603), the X domain and the Y domain would be bridged. This bridging would activate a transcriptase that begins transcribing a unique RNA sequence (607) that would identify a particular target molecule. The unique sequence would correspond to a target molecule. The unique RNA sequence would have P5 and P7 adaptor sequences flanking the sequence. The transcripts would be collected and analyzed to detect their presence, allowing quantification of the target molecule.

The P5 and P7 adaptor sequences would bind to the flow cell of the Illumina platform, allowing for the bridge amplification needed for sequencing. This would allow detection and quantification of the molecular target.

### REFERENCES

Additional background information can be found in the following references, each of which is hereby incorporated by reference in its entirety.
singerinstruments.com/resource/yeast-2-hybrid/
Brückner A, Polge C, Lentze N, Auerbach D, Schlattner U. Yeast two-hybrid, a powerful tool for systems biology.
Int J Mol Sci. 2009 Jun. 18;10(6):2763-88. doi: 10.3390/ijms10062763. PMID: 19582228; PMCID: PMC2705515.
Vaish N K, Jadhav V R, Kossen K, Pasko C, Andrews L E, McSwiggen J A, Polisky B, Seiwert S D. Zeptomole detection of a viral nucleic acid using a target-activated ribozyme. RNA. 2003 Sep;9(9):1058-72. doi: 10.1261/rna.5760703. PMID: 12923255; PMCID: PMC1370471.
Cui, N., Zhang, H., Schneider, N. et al. A mix-and-read drop-based in vitro two-hybrid method for screening high-affinity peptide binders. Sci Rep 6, 22575 (2016). https://doi.org/10.1038/srep22575
Kossen K, Vaish N K, Jadhav V R, Pasko C, Wang H, Jenison R, McSwiggen J A, Polisky B, Seiwert S D. High-throughput ribozyme-based assays for detection of viral nucleic acids. Chem Biol. 2004 Jun; 11(6):807-15. doi: 10.1016/j.chembiol.2004.03.029. PMID: 15217614.

Having described the embodiments in detail, it will be apparent that modifications, variations, and equivalent embodiments are possible without departing the scope of the defined in the appended claims. Furthermore, it should be appreciated that all examples in the present disclosure are provided as non-limiting examples.

The above non-limiting methods and examples are provided to further illustrate the embodiments disclosed herein. It should be appreciated by those of skill in the art that the techniques disclosed in the methods and examples that follow represent approaches that have been found to function well in practice, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the embodiments.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

1. A method comprising:
(i) bridging a first molecule domain with a second molecule domain by a target molecule, wherein the bridging activates transcription of one or more ribozymes;
(ii) interacting a probe with the one or more ribozymes to produce an RNA readout; and
(iii) detecting the RNA readout.

2. A method comprising:
(i) partitioning a reaction mixture within a microfluidic device into partitions, the reaction mixture comprising:
a. one or more target molecules;
b. one or more first molecule domains, the first molecule domains capable of interacting with one or more target molecules;
c. one or more second molecule domains, the second molecule domains capable of interacting with one or more target molecules and one or more first molecule domains by bridging the first molecule domain to the second molecule domain, where bridging of the first molecule domain to the second molecule domain by interacting with the target molecules, transcribes one or more ribozymes;
(ii) reacting the reaction mixture to produce reacted partitions within the microfluidic device with one or more probes, wherein the one or more probes interact with one or more ribozymes to produce an RNA readout;
(iii) detecting the RNA readout; and
(iv) quantifying the one or more target molecules from the reacted partitions,

3. The method of claim 1, wherein the target molecule is added to a reaction mixture comprising a first molecule domain and a second molecule domain.

4. The method of claim 1, further comprising quantifying the RNA readout to determine a quantity of the target molecule.

5. The method of claim 2, further comprising collecting the reacted partitions from the microfluidic device.

6. The method of claim 2, further comprising quantifying the ribozymes as a readout to determine the quantity of target molecules.

7. The method of claim 1, wherein the target molecule comprises at least one of a protein, a nucleic acid, a carbohydrate, a lipid, a hormone, a signaling molecule, or any combination thereof.

8. (canceled)

9. The method of claim 1, wherein the method detects single target molecules.

10-12. (canceled)

13. The method of claim 1, wherein the first molecule domain is linked to a DNA binding domain capable of interacting with an enhancer region, the enhancer region capable of activating transcription a gene encoding a ribozyme.

14. (canceled)

15. The method of claim 1, wherein the second molecule domain is linked to a transcriptional activation domain capable of interacting with a transcriptional region on DNA sequence.

16. (canceled)

17. The method of claim 1, wherein the first binding domain, the second binding domain, or both, bind to an antibody, aptamer, or combination thereof.

18-22. (canceled)

23. The method of claim 1, wherein the first binding domain is an antibody, aptamer, or combination thereof.

24. The method of claim 1, wherein the second binding domain is an antibody, aptamer, or combination thereof.

25. The method of claim 1, wherein the ribozyme is selected from a GIR1 branching ribozyme, glmS ribozyme, Group I self-splicing intron,
Group II self-splicing intron, Hairpin ribozyme, Hammerhead ribozyme, HDV ribozyme, rRNA, RNase P, Twister ribozyme, Twister sister ribozyme, VS ribozyme, Pistol ribozyme, Hatchet ribozyme, Viroids, Deoxyribozyme, or an artificial ribozyme.

26. The method of claim 1, wherein the interaction of the probe with one or more ribozymes comprises a ligation, cleavage, binding, chemical modification, or any combination thereof of the probe.

27. The method of claim 1, wherein the RNA readout is a fluorescent readout.

28. The method of claim 1, wherein the probes comprise an oligonucleotides, peptides, or combinations thereof.

29-35. (canceled)

36. A method of diagnosis of a disease or condition in a subject in need thereof comprising use of the method of claim 1 to detect the target molecules indicating a disease or condition in the subject.

37. A method of treating a disease or condition in a subject in need thereof comprising use of the method of claim 1 any of claims 1-35 to detect the target molecules indicating the disease or condition in the subject.

38. (canceled)

39. The method of claim 36, wherein the disease or condition comprise diseases caused by prions, heart disease, cancer genetic diseases, or combinations thereof.

40-43. (canceled)

44. A kit comprising:
(i) a DNA sequence comprising:
an enhancer region, the enhancer region capable of interacting with a DNA binding domain;
a transcriptional region, capable of interacting with a transcriptional activation domain, wherein the transcriptional region encodes a ribozyme;
(ii) one or more first molecule domains, capable of interacting with a target molecule;
(iii) one or more second molecule domains, capable of interacting with a target molecule; and
(iv) one or more probes capable of interacting with one or more ribozymes to produce an RNA readout.

45-48. (canceled)
